(19) United States
(12) Patent Application Publication
(10) Pub. No.: US 2011/0136872 A1
(43) Pub. Date: Jun. 9, 2011

(54) STABLE AQUEOUS COMPOSITIONS OF PROSTGLANDIN AGONIST PRODRUGS AND METHODS FOR USE THEREOF

(76) Inventor: Robert M. Burk, Laguna Beach, CA (US)

(21) Appl. No.: 12/939,861

(22) Filed: Nov. 4, 2010

Related U.S. Application Data
(60) Provisional application No. 61/267,897, filed on Dec. 9, 2009.

(51) Int. Cl. A61K 31/4436; A61K 31/4025; A61P 27/02; A61P 27/10

(52) U.S. Cl. 514/336; 514/422

(57) ABSTRACT

The present invention is based on the discovery that a marked increase in aqueous stability (and thereby shelf life) of prostanoid agonist prodrug compositions is achieved by incorporating into the compositions certain well-defined carboxylic acids, and thereafter adjusting the pH of the compositions from about 4.0 to about 8.0. As a result, the compositions and methods of the invention provide the aqueous stability required for marketable topical drug treatments of a wide variety of ocular disorders.

### RELATED APPLICATION

This application claims the benefit of U.S. Provisional Application Ser. No. 61/267,897, filed Dec. 9, 2009, the disclosure of which is hereby incorporated in its entirety herein by reference.

### FIELD OF THE INVENTION

The present invention relates generally to compositions of prodrugs of prostanoid agonists and more specifically to stable aqueous compositions of the prostanoid agonists prodrugs and methods for use thereof.

### BACKGROUND OF THE INVENTION

Ocular hypotensive agents are useful in the treatment of a number of various ocular hypertensive conditions, such as post-surgical and post-laser trabeculectomy ocular hypertensive episodes, glaucoma, and as presurgical adjuncts.

Glaucoma is a disease of the eye characterized by increased intraocular pressure. On the basis of its etiology, glaucoma has been classified as primary or secondary. For example, primary glaucoma in adults (congenital glaucoma) may be either open-angle or acute or chronic angle-closure. Secondary glaucoma results from pre-existing ocular diseases such as uveitis, intraocular tumor or an enlarged cataract.

The underlying causes of primary glaucoma are not yet known. The increased intraocular tension is due to the obstruction of aqueous humor outflow. In chronic open-angle glaucoma, the anterior chamber and its anatomic structures appear normal, but drainage of the aqueous humor is impeded. In acute or chronic angle-closure glaucoma, the anterior chamber is shallow, the filtration angle is narrowed, and the iris may obstruct the trabecular meshwork at the entrance of the canal of Schlemm. Dilation of the pupil may push the root of the iris forward against the angle, and may produce pupilary block and thus precipitate an acute attack. Eyes with narrow anterior chamber angles are predisposed to acute angle-closure glaucoma attacks of various degrees of severity.

Secondary glaucoma is caused by any interference with the flow of aqueous humor from the posterior chamber into the anterior chamber and subsequently, into the canal of Schlemm. Inflammatory disease of the anterior segment may prevent aqueous escape by causing complete posterior synechia in iris bombe, and may plug the drainage channel with exudates. Other common causes are intraocular tumors, enlarged cataracts, central retinal vein occlusion, trauma to the eye, operative procedures and intraocular hemorrhage.

Considering all types together, glaucoma occurs in about 2% of all persons over the age of 40 and may be asymptotic for years before progressing to rapid loss of vision. In cases where surgery is not indicated, topical-adrenoreceptor antagonists have traditionally been the drugs of choice for treating glaucoma.

Certain eicosanoids and their derivatives have been reported to possess ocular hypotensive activity, and have been recommended for use in glaucoma management. Eicosanoids and derivatives include numerous biologically important compounds such as prostanoids and their derivatives. Prostanoids can be described as derivatives of prostanoic acid which have the following structural formula:

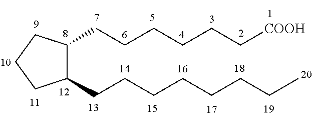

Various types of prostanoids are known, depending on the structure and substituents carried on the alicyclic ring of the prostanoic acid skeleton. Further classification is based on the number of unsaturated bonds in the side chain indicated by numerical subscripts after the generic type of prostanoid (e.g. prostanoid E₁ (PGE₁), prostanoid E₂ (PGE₂)), and on the configuration of the substituents on the alicyclic ring indicated by or (e.g. prostanoid F₂ (PGF₂)].

Prostanoids were earlier regarded as potent ocular hypertensives, however, evidence accumulated in the last two decades shows that some prostanoids are highly effective ocular hypotensive agents, and are ideally suited for the long-term medical management of glaucoma (see, for example, Bito, L. Z. Biological Protection with Prostanoids, Cohen, M. M., ed., Boca Raton, Fla., CRC Press Inc., 1985, pp. 231-252; and Bito, L. Z., Applied Pharmacology in the Medical Treatment of Glaucomas Drance, S. M. and Neufeld, A. H. eds., New York, Grune & Stratton, 1984, pp. 477-505. Such prostanoids include PGF₂ PGF₁, PGE₂, and certain lipid-soluble esters, such as C₁ to C₂ alkyl esters, e.g. 1-isopropyl ester, of such compounds.

Although the precise mechanism is not yet known experimental results indicate that the prostanoid-induced reduction in intraocular pressure results from increased uveoscleral outflow (Nilsson et. al., Invest. Ophthalmol. Vis. Sci. (suppl), 284 (1987)).

The isopropyl ester of PGF₂ has been shown to have significantly greater hypotensive potency than the parent compound, presumably as a result of its more effective penetration through the cornea. In 1987, this compound was described as “the most potent ocular hypotensive agent ever reported” [see, for example, Bito, L. Z., Arch. Ophthalmol. 105, 1036 (1987), and Siebold et al., Prodrug 5 3 (1989)].

Whereas prostanoids appear to be devoid of significant intraocular side effects, ocular surface (conjunctival) hyperemia and foreign-body sensation have been consistently associated with the topical ocular use of such compounds, in particular PGF₂ and its prodrugs, e.g., its 1-isopropyl ester, in humans. The clinical potentials of prostanoids in the management of conditions associated with increased ocular pressure, e.g. glaucoma are greatly limited by these side effects.

In a series of United States patents assigned to Allergan, Inc. prostanoid esters with increased ocular hypotensive activity accompanied with no or substantially reduced side-effects are disclosed. Some representative examples are U.S. Pat. No. 5,446,041, U.S. Pat. No. 4,994,274, U.S. Pat. No. 5,028,624 and U.S. Pat. No. 5,034,413 all of which are hereby expressly incorporated by reference.

Further pertinent background information is provided regarding the term “prodrug”. An ester is a compound which is converted to a therapeutically active compound after administration, and the term should be interpreted as broadly herein as is generally understood in the art. While not intending to limit the scope of the invention, conversion may occur by hydrolysis of an ester group or some other biologically labile group. Generally, but not necessarily, an ester is inactive or less active than the therapeutically active compound to which it is converted.

### SUMMARY OF THE INVENTION

The present invention is based on the discovery that a marked increase in aqueous stability (and thereby shelf life) of prostanoid agonist compositions is achieved by incorporating into the compositions certain well-defined carboxylic acids, and thereafter adjusting the pH of the compositions from about 4.0 to about 8.0. As a result, the compositions and methods of the invention provide the aqueous stability required for marketable topical drug treatments of a wide variety of ocular disorders.

In one embodiment of the invention, there are provided compositions including an ester of a prostanoid agonist, a carboxylic acid, sodium phosphate dibasic, sodium chloride, a solubilizing agent, and the remainder water, wherein the pH of the composition is adjusted from about 4 to about 8.

In another embodiment of the invention, there are provided methods for conferring aqueous stability to a composition including an ester of a prostanoid agonist. Such methods can be performed, for example, by adding a carboxylic acid to the composition and thereby adjusting the pH to from 4 to about 8.

In another embodiment of the invention, there are provided methods for treating an ocular disorder. Such methods can be performed, for example, by administering to a subject in need thereof a therapeutically effective amount of a composition including an ester of a prostanoid agonist, a carboxylic acid, sodium phosphate dibasic, sodium chloride, a solubilizing agent, and the remainder water, wherein the pH of the composition is adjusted from about 4 to about 8.

### DETAILED DESCRIPTION OF THE INVENTION

It is to be understood that both the foregoing general description and the following detailed description are exemplary and explanatory only and are not restrictive of the invention claimed. As used herein, the use of the singular includes the plural unless specifically stated otherwise. As used herein, “or” means “and/or” unless stated otherwise. Furthermore, use of the term “including” as well as other forms, such as “includes,” and “included,” is not limiting. The section headings used herein are for organizational purposes only and are not to be construed as limiting the subject matter described.

Unless specific definitions are provided, the nomenclatures utilized in connection with, and the laboratory procedures and techniques of analytical chemistry, synthetic organic and inorganic chemistry described herein are those known in the art. Standard chemical symbols are used interchangeably with the full names represented by such symbols. Thus, for example, the terms “hydrogen” and “H” are understood to have identical meaning. Standard techniques may be used for chemical syntheses, chemical analyses, and formulation.

As used herein, “alkyl” refers to straight or branched chain hydrocarbyl groups having from 1 up to about 100 carbon atoms. Whenever it appears herein, a numerical range, such as “1 to 100” or “C₁-C₁₀₀”, refers to each integer in the given range; e.g., “C₁-C₁₀₀ alkyl” means that an alkyl group may comprise only 1 carbon atom, 2 carbon atoms, 3 carbon atoms, etc., up to and including 100 carbon atoms, although the term “alkyl” also includes instances where no numerical range of carbon atoms is designated. “Substituted alkyl” refers to alkyl moieties bearing substituents including alkyl, alkenyl, alkynyl, hydroxy, oxo, alkoxy, mercapto, cycloalkyl, substituted cycloalkyl, heterocyclic, substituted heterocyclic, aryl, substituted aryl, heteroaryl, substituted heteroaryl, aryloxy, substituted aryloxy, halogen, haloalkyl, cyano, nitro, nitrone, amino, lower alkylamino, lower alkyldiamino, amido, azido, —C(O)H, —C(O)R₇, —CH₂OR₇, —C(O)—, —C(O)—, —S—, —S(O)₂, —OC(O)—O—, wherein R₇ is H or lower alkyl, acyl, oxyacyl, carboxyl, carbamate, sulfonyl, sulfonamide, sulfuryl, and the like. As used herein, “lower alkyl” refers to alkyl moieties having from 1 to about 6 carbon atoms.

As used herein, “cycloalkyl” refers to cyclic (i.e., ring-containing) alkyl moieties typically containing in the range of about 3 up to about 8 carbon atoms, and “substituted cycloalkyl” refers to cycloalkyl groups further bearing one or more substituents as set forth above.

As used herein, “alkenyl” refers to straight or branched chain hydrocarbyl groups having at least one carbon-carbon double bond, and having in the range of about 2 up to about 100 carbon atoms, and “substituted alkenyl” refers to alkenyl groups further bearing one or more substituents as set forth above. As used herein, “lower alkenyl” refers to alkenyl moieties having from 2 to about 6 carbon atoms

As used herein, “oxyalkyl” refers to an alkyl moiety wherein at least one methylene unit has been replaced by an oxygen atom.

As used herein, “oxyalkenyl” refers to an alkenyl moiety wherein at least one methylene unit has been replaced by an oxygen atom.

As used herein, “hydroxyalkyl” refers to an alkyl moiety bearing at least one hydroxyl group.

As used herein, “hydroxyalkenyl” refers to an alkenyl moiety bearing at least one hydroxyl group.

As used herein, “arylene” refers to divalent aromatic groups having in the range of 6 up to 14 carbon atoms and “substituted arylene” refers to divalent aryl groups further bearing one or more substituents as set forth above

As used herein, “heteroarylene” refers to aromatic moieties containing one or more heteroatoms (e.g., N, O, S, or the like) as part of the ring structure and having in the range of 5 up to 14 total atoms in the ring structure (i.e., carbon atoms and heteroatoms). “Substituted heteroarylene” refers to heteroarylene groups further bearing one or more substituents as set forth above.

As used herein, “halogen” or “halide” refers to fluoride, chloride, bromide or iodide.

The invention provides compositions including an ester of a prostanoid agonist, a carboxylic acid, sodium phosphate dibasic, sodium chloride, a solubilizing agent, and the remainder water, wherein the pH of the composition is adjusted from about 4 to about 8. In some embodiments, the pH of the composition is adjusted to from about 4.5 to about 6.5. In one embodiment, the pH of the composition is adjusted to about 6.0.

The compositions described herein exhibit remarkable aqueous stability, thereby resulting in increased shelf life for a pharmaceutical formulation containing invention compositions.

As used herein, the phrase “aqueous stability” means

In certain embodiments of the invention, ester prodrugs of the prostanoid agonists disclosed herein are contemplated. An ester may be derived from a carboxylic acid of C1 (i.e. the terminal carboxylic acid of a natural prostanoid), or an ester may be derived from a carboxylic acid functional group on another part of the molecule, such as on a phenyl ring. While not intending to be limiting, an ester may be an alkyl ester, an aryl ester, or a heteroaryl ester. In some embodiments, C₁₋₆ alkyl esters are contemplated for use in the practice of the invention, wherein the alkyl part of the ester has from 1 to 6 carbon atoms and includes, but is not limited to, methyl, ethyl, propyl, isopropyl, n-butyl, sec-butyl, iso-butyl, t-butyl, pentyl isomers, hexyl isomers, cyclopropyl, cyclobutyl, cyclopentyl, cyclohexyl, and combinations thereof having from 1-6 carbon atoms, etc.

Prostanoid agonist prodrugs contemplated for use in the compositions of the invention have the structure:

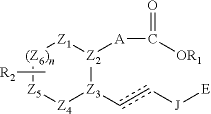

wherein:
   each of Z₁ to Z₆ is independently C, N, O, or S;
   A is —(CH₂)₆—, or cis-CH₂CH═CH—(CH₂)₃—, wherein 1 or 2 carbons may be substituted with S or O; or
   A is —(CH₂)ₘ—Ar—(CH₂)ₒ— wherein Ar is arylene or heteroarylene, the sum of m and o is from 1 to 4, and wherein one CH₂ may be substituted with S or O;
   R₁ is alkyl, cycloalkyl, oxyalkyl, hydroxyalkyl, alkenyl, oxyalkenyl, or hydroxyalkenyl;
   R₂ is alkyl, hydroxyl, halide, or oxo;
   J is alkyl, cycloalkyl, oxyalkyl, hydroxyalkyl;
   E is C₁₋₁₂ alkyl, R₃, or —Y—R₃ wherein Y is CH₂, S, or O, and R₃ is aryl or heteroaryl;
   n is 0 or 1;
   and wherein a dashed line represents the presence or absence of a bond.

In some embodiments, the prostanoid agonist prodrugs have the structure wherein n is 0.

In other embodiments, the prostanoid agonist prodrugs have the structure wherein R₁ is alkyl or hydroxyalkyl. In certain embodiments, R₁ is isopropyl or —CH₂—CH₂—OH.

Exemplary prostanoid agonist prodrugs include, but are not limited to, compounds having the structure:

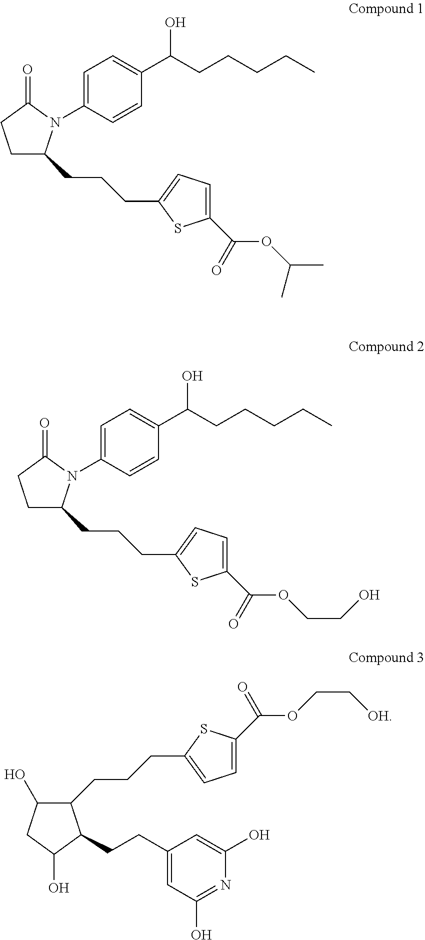

A wide range of carboxylic acids are contemplated for use in the compositions of the invention. In some embodiments, the carboxylic acid is a C₁ to C₁₀ carboxylic acid. In one embodiment, the carboxylic acid is citric acid.

The carboxylic acid is typically present in the composition at a concentration of about 0.05 wt % to about 0.2 wt %. In some embodiments, the carboxylic acid is present in the composition at a concentration of about 0.1 wt % to about 0.15 wt %. In one embodiment, the carboxylic acid is present in the composition at a concentration of 0.135 wt % carboxylic acid.\

Sodium phosphate dibasic is typically present in the composition at a concentration of about 1.0 wt % to about 2.0 wt %. In some embodiments, sodium phosphate dibasic is present in the composition at a concentration of about 1.2 wt % to about 1.6 wt %. In one embodiment, sodium phosphate dibasic is present in the composition at a concentration of about 1.42 wt %

Sodium chloride is typically present in the composition at a concentration of about 0.05 wt % to about 0.2 wt %. In some embodiments, sodium chloride is present in the composition at a concentration of about 0.1 wt % to about 0.15 wt %. In one embodiment, sodium chloride is present in the composition at a concentration of about 0.135 wt %.

A wide variety of solubilizing agents are contemplated for use in the practice of the invention, such as for example, polysorbate 80, pluronic F127, and the like.

In another embodiment of the invention, there are provided methods for conferring aqueous stability to a formulation comprising an ester of a prostanoid agonist. Such methods are performed, for example, by adding a carboxylic acid to the formulation and thereby adjusting the pH to from 4 to about 8. In some embodiments, the pH is adjusted from about 4.5 to about 6.5. In some embodiments, the pH is adjusted to about 6.0.

In other embodiments of the invention, there are provided methods for treating an ocular disorder. Such methods can be performed, for example, by administering to a subject in need thereof a therapeutically effective amount of a composition including an ester of a prostanoid agonist, a carboxylic acid, sodium phosphate dibasic, sodium chloride, a solubilizing agent, and the remainder water, wherein the pH of the composition is adjusted from about 4 to about 8.

As used herein, the term “therapeutically effective amount” means the amount of the pharmaceutical composition that will elicit the biological or medical response of a subject in need thereof that is being sought by the researcher, veterinarian, medical doctor or other clinician. In some embodiments, the subject in need thereof is a mammal. In some embodiments, the mammal is human.

Disorders that can be treated using the methods of the invention include, but are not limited to, glaucoma, elevated intraocular pressure, optic neuropathy, corneal pain, diabetic retinopathy, retinal dystrophies, macular degeneration, non-exudative age related macular degeneration (ARMD), exudative Age Related Macular Degeneration (ARMD), Lebers optic neuropathy, optic neuritis often associated with multiple sclerosis, retinal vein occlusions, ischemic neuropathies and other neurodegenerative diseases, choroidal neovascularization, central serous chorioretinopathy, cystoid macular edema, diabetic macular edema, myopic retinal degeneration, acute multifocal placoid pigment epitheliopathy, Behcet's disease, birdshot retinochoroidopathy, intermediate uveitis (pars planitis), multifocal choroiditis, multiple evanescent white dot syndrome (MEWDS), ocular sarcoidosis, posterior scleritis, serpiginous choroiditis, subretinal fibrosis and uveitis syndrome, Vogt-Koyanagi-Harada syndrome, punctate inner choroidopathy, acute posterior multifocal placoid pigment epitheliopathy, acute retinal pigment epitheliitis, acute macular neuroretinopathy, and following procedures such as photodynamic therapy and laser-assisted in situ keratomileusis (LASIK).

The following examples are intended only to illustrate the invention and should in no way be construed as limiting the invention.

### EXAMPLES

The aqueous stability of invention compositions was evaluated using Compounds 2 and 3. Four formulations were prepared for each compound, as set forth in the tables below.

TABLE 1

| | Formulation | Formulation | Formulation | Formulation |
|---|---|---|---|---|
| Wt % | 1 | 2 | 3 | 4 |
| Compound 2 | 0.01 | 0.01 | 0.01 | 0.01 |
| Sodium | 1.42 | 1.42 | 1.42 | 1.42 |
| phosphate | | | | |
| dibasic, | | | | |
| anhydrous | | | | |
| Citric acid | 0.136 | 0.136 | 0.136 | 0.136 |
| Sodium | 0.12 | 0.12 | 0.12 | 0.12 |
| chloride | | | | |
| Polysorbate 80 | 1.0 | 1.0 | | |
| Pluronic F127 | | | 1.0 | 1.0 |
| pH | 6.0 | 7.3 | 6.0 | 7.3 |

TABLE 2

| | Formulation | Formulation | Formulation | Formulation |
|---|---|---|---|---|
| Wt % | 1 | 2 | 3 | 4 |
| Compound 3 | 0.01 | 0.01 | 0.01 | 0.01 |
| Sodium | 1.42 | 1.42 | 1.42 | 1.42 |
| phosphate | | | | |
| dibasic, | | | | |
| anhydrous | | | | |
| Citric acid | 0.136 | 0.136 | 0.136 | 0.136 |
| Sodium | 0.12 | 0.12 | 0.12 | 0.12 |
| chloride | | | | |
| Polysorbate 80 | 1.0 | 1.0 | | |
| Pluronic F127 | | | 1.0 | 1.0 |
| pH | 6.0 | 7.3 | 6.0 | 7.3 |

The formulations were analyzed by HPLC with the following measurement parameters:

Column: BioWidePore C18 (SUPELCO), 4.6 mm×25 cm, 5 μm
Mobile Phase A: 0.1% (V/V) trifluoroacetic acid (TFA) in di-water, 0.8 micron filtered
Mobile Phase B: 100% acetonitrile, 0.8 micron filtered
Column temp: Ambient
Injection volume: 30 μL

### UV Detection: 214 nm

Flow: 1.0 mL/min
Run time: 25 minutes
Sample diluent: 50% acetonitrile in di-water

Using the above HPLC parameters, the following stability data was generated:

TABLE 3
Compound 1, Formulation 1

| % Recovery | 30° C. | 45° C. | 60° C. |
|---|---|---|---|
| 15 days | 99.8 | 98.3 | 89.1 |
| 30 days | 99.4 | 98.5 | 88.3 |
| 45 days | 100.3 | 98.1 | 78.7 |

TABLE 4
Compound 1, Formulation 2

| % Recovery | 30° C. | 45° C. | 60° C. |
|---|---|---|---|
| 15 days | 100.7 | 95.7 | 89.5 |
| 30 days | 99.9 | 96.0 | 85.8 |
| 45 days | 101.7 | 97.9 | 81.1 |

TABLE 5
Compound 1, Formulation 3

| % Recovery | 30° C. | 45° C. | 60° C. |
|---|---|---|---|
| 15 days | 101.5 | 101.5 | 92.3 |
| 30 days | 99.7 | 103.1 | 55.1 |
| 45 days | 96.7 | 98.2 | 51.3 |

TABLE 6
Compound 1, Formulation 4

| % Recovery | 30° C. | 45° C. | 60° C. |
|---|---|---|---|
| 15 days | 96.9 | 100.4 | 85.1 |
| 30 days | 93.2 | 88.6 | 36.0 |
| 45 days | 93.7 | 89.9 | 33.4 |

TABLE 7
Compound 2, Formulation 1

| % Recovery | 30° C. | 45° C. | 60° C. |
|---|---|---|---|
| 15 days | 97.7 | 100.2 | 93.8 |
| 30 days | 99.6 | 99.1 | 86.8 |
| 45 days | 100.6 | 98.5 | 80.8 |

TABLE 8
Compound 2, Formulation 2

| % Recovery | 30° C. | 45° C. | 60° C. |
|---|---|---|---|
| 15 days | 101.8 | 100.4 | 94.4 |
| 30 days | 101.6 | 99.4 | 86.8 |
| 45 days | 100.8 | 96.7 | 81.3 |

TABLE 9
Compound 2, Formulation 3

| % Recovery | 30° C. | 45° C. | 60° C. |
|---|---|---|---|
| 15 days | 103.2 | 103 | 100.2 |
| 30 days | 106.6 | 103.3 | 82.9 |
| 45 days | 105.8 | 101.1 | 79.3 |

TABLE 10
Compound 2, Formulation 4

| % Recovery | 30° C. | 45° C. | 60° C. |
|---|---|---|---|
| 15 days | 101.7 | 100.7 | 94.6 |
| 30 days | 102.9 | 100.2 | 73.6 |
| 45 days | 102.4 | 99.6 | 62.3 |

From the above stability data, it is apparent that at 30° C. both test Compounds 2 and 3 are stable in each formulation for 45 days. At 45° C., no significant loss was seen in most formulations, with the exception of Compound 2 in Formulation 4.

In addition, it can be concluded that Formulation 2 is superior for both test compounds, and that polysorbate 80 formulations appear to be superior to the pluronic F127 formulations. Finally, both test compounds appear to be more stable at pH 6 than at pH 7.3.

While this invention has been described with respect to these specific examples, it is understood that other modifications and variations are possible without departing from the spirit of the invention.

What is claimed is:

1. A composition comprising an ester of a prostanoid, a carboxylic acid, sodium phosphate dibasic, sodium chloride, a solubilizing agent, and the remainder water, wherein the pH of the composition is adjusted from about 4 to about 8.

2. The composition of claim 1, wherein the carboxylic acid is a C₁ to C₁₀ carboxylic acid.

3. The composition of claim 1, wherein the carboxylic acid is citric acid.

4. The composition of claim 1 having about 0.05% to about 0.2% carboxylic acid.

5. The composition of claim 1 having about 0.1% to about 0.15% carboxylic acid.

6. The composition of claim 1 having 0.135% carboxylic acid.

7. The composition of claim 1 having a pH from about 4.5 to about 6.5.

8. The composition of claim 1 having a pH of about 6.0.

9. The composition of claim 1, wherein the prodrug of the prostanoid agonist has the structure: 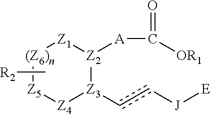
wherein:
each of Z₁ to Z₆ is independently C, N, O, or S;
A is —(CH₂)₆—, or cis-CH₂CH═CH—(CH₂)₃—, wherein 1 or 2 carbons may be substituted with S or O; or
A is —(CH₂)ₘ—Ar—(CH₂)ₒ— wherein Ar is arylene or heteroarylene, the sum of m and o is from 1 to 4, and wherein one CH₂ may be substituted with S or O;
R₁ is alkyl, cycloalkyl, oxyalkyl, hydroxyalkyl, alkenyl, oxyalkenyl, or hydroxyalkenyl;
R₂ is alkyl, alkenyl, hydroxyl, halide, cyano, or oxo;
J is alkyl, cycloalkyl, oxyalkyl, hydroxyalkyl;
E is C₁₋₁₂ alkyl, R₃, or —Y—R₃ wherein Y is CH₂, S, or O, and R₃ is aryl or heteroaryl;
n is 0 or 1;
and wherein a dashed line represents the presence or absence of a bond.

10. The composition of claim 9, wherein n is 0.

11. The composition of claim 9, wherein R₁ is alkyl or hydroxyalkyl.

12. The composition of claim 9, wherein R₁ is isopropyl or —CH₂—CH₂—OH.

13. The composition of claim 9, wherein the prodrug of the prostanoid agonist has the structure:

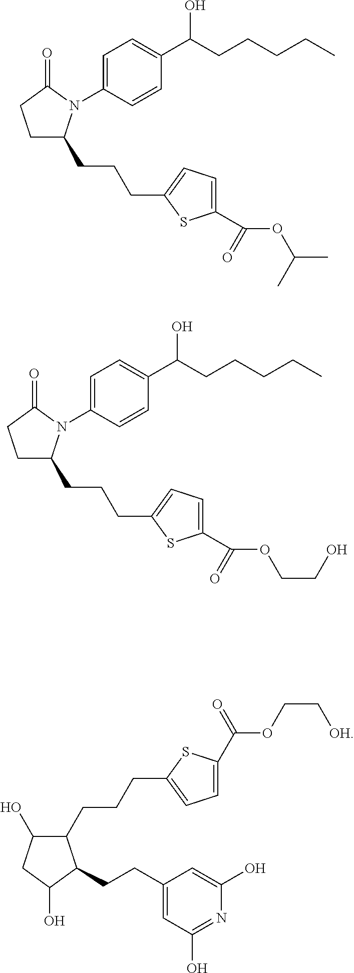

14. The composition of claim 1, having about 1.0% to about 2.0% sodium phosphate dibasic.

15. The composition of claim 1, having about 1.2% to about 1.6% sodium phosphate dibasic.

16. The composition of claim 1, having about 1.42% sodium phosphate dibasic.

17. The composition of claim 1 having about 0.05% to about 0.2% sodium chloride.

18. The composition of claim 1 having about 0.1% to about 0.15% sodium chloride.

19. The composition of claim 1 having 0.135% sodium chloride.

20. The composition of claim 1, wherein the solubilizing agent is polysorbate 80 or pluronic F127.

21. A method for conferring aqueous stability to a formulation comprising an ester of a prostanoid agonist, comprising adding a carboxylic acid to the formulation and thereby adjusting the pH to from 4 to about 8.

22. The method of claim 21 wherein the pH is adjusted from about 4.5 to about 6.5.

23. The method of claim 21 wherein the pH is adjusted to about 6.0.

24. The method of claim 21 wherein the carboxylic acid is citric acid.

25. A method for treating an ocular disorder comprising administering to a subject in need thereof a therapeutically effective amount of a composition according to claim 1.

26. The method of claim 25 wherein the disorder is glaucoma, elevated intraocular pressure, optic neuropathy, corneal pain, diabetic retinopathy, retinal dystrophies, macular degeneration, non-exudative age related macular degeneration (ARMD), exudative Age Related Macular Degeneration (ARMD), Lebers optic neuropathy, optic neuritis often associated with multiple sclerosis, retinal vein occlusions, ischemic neuropathies and other neurodegenerative diseases, choroidal neovascularization, central serous chorioretinopathy, cystoid macular edema, diabetic macular edema, myopic retinal degeneration, acute multifocal placoid pigment epitheliopathy, Behcet's disease, birdshot retinochoroidopathy, intermediate uveitis (pars planitis), multifocal choroiditis, multiple evanescent white dot syndrome (MEWDS), ocular sarcoidosis, posterior scleritis, serpiginous choroiditis, subretinal fibrosis and uveitis syndrome, Vogt-Koyanagi-Harada syndrome, punctate inner choroidopathy, acute posterior multifocal placoid pigment epitheliopathy, acute retinal pigment epitheliitis, acute macular neuroretinopathy, and following procedures such as photodynamic therapy and laser-assisted in situ keratomileusis (LASIK).

27. The method of claim 25 wherein the disorder is glaucoma or elevated intraocular pressure.
